(19) United States
(12) Patent Application Publication
(10) Pub. No.: US 2016/0144122 A1
(43) Pub. Date: May 26, 2016

(54) Syringe Assembly with Plunger Rod Backstop and Method of Use

(71) Applicant: Fisher Clinical Services GmbH, Allschwil (CH)

(72) Inventors: Patrick Locati, Binningen (CH); Guido Hunkeler, Schonenbuch (CH)

(21) Appl. No.: 14/555,039

(22) Filed: Nov. 26, 2014

(51) Int. Cl. A61M 5/315

(52) U.S. Cl. CPC A61M 5/31501; A61M 2005/31508; A61M 2202/0007; A61M 2207/00

(57) ABSTRACT

A syringe assembly includes a syringe barrel having a tubular body that bounds a compartment extending between a proximal end and an opposing distal end and a flange that radially outwardly projects from the proximal end of the body. A stopper is movably disposed with the compartment of the syringe barrel. A plunger rod includes a shaft having a proximal end and an opposing distal end and a plurality of teeth disposed on the shaft, the distal end of the shaft being secured to the stopper. A backstop engages the flange of the syringe barrel and at least a portion of the plurality of teeth of the plunger rod.

### CROSS-REFERENCE TO RELATED APPLICATIONS

Not applicable.

### BACKGROUND OF THE INVENTION

1. The Field of the Invention

The present invention relates to syringe assemblies having a plunger rod backstop and related methods of use.

2. The Relevant Technology

Clinical trials for some pharmaceutical drugs require that the drug dosage be delivered by injection through the use of a syringe. As part of the clinical trial protocols, the syringes are preloaded with a single dose of the drug or a placebo prior to shipping and use. The plunger rod for each syringe is typically attached to a stopper within the syringe barrel prior to shipping. Precautions are taken to ensure that there is no excessive movement of the stopper relative to the syringe barrel during transport of the preloaded syringes. Excessive movement of the stopper relative to the syringe barrel can potentially result in contamination of the dosage.

Movement of the stopper during transport can be a result of a change in atmospheric pressure. For example, the portion of the syringe barrel containing the dosage will also typically contain an air bubble. If the preloaded syringes are being transported by aircraft, the decrease in atmospheric pressure during the flight can cause the gas bubble to expand. In turn, expansion of the gas bubble can result in movement of stopper which, in turn, can potentially result in the dosage being contaminated.

In one attempt to preclude movement of the stopper during transport of the preloaded syringes, each preloaded syringe is housed within a separate, specially designed packaging case. The packaging case holds the plunger rod fixed relative to the syringe barrel so that neither the plunger rod nor the stopper can move relative to the syringe barrel when the preloaded syringe is subject to a change in atmospheric pressure.

Although the packaging cases are useful in preventing unwanted movement of the plunger rod and stopper, they have a number of shortcomings. For example, the packing cases are relatively large and must be reinforced to withstanding the applied pressures. As such, the packaging cases are a significant expense to produce, transport and store. Furthermore, the packaging cases are designed for a specific syringe configuration having a specific dose size. Accordingly, different packaging cases must be made for different syringes and for different dose sizes. This requirement adds to the cost and complexity of packaging and distribution of the preloaded syringes. Other shortcomings also exist.

Accordingly, what is needed in the art are syringe assemblies that overcome all or some of the above shortcomings.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a perspective view of an inventive syringe assembly incorporating features of the present invention;

FIG. 2 is a partially exploded view of the syringe assembly depicted in FIG. 1;

FIG. 3 is a perspective view of the stopper of the syringe assembly depicted in FIG. 1;

FIG. 4 is a perspective view of the distal side of the backstop of the syringe assembly depicted in FIG. 1;

FIG. 5 is a perspective view of the proximal side of the backstop depicted in FIG. 4;

FIG. 6 is an elevated view of the distal side of the backstop shown in FIG. 4;

FIG. 7 is a cross sectional top view of the pawls of the backstop shown in FIG. 5;

FIG. 8 is a perspective view of the syringe assembly shown in FIG. 1 partially assembled;

FIG. 9 is a top plan view of the syringe assembly shown in FIG. 1 showing the backstop engaging the syringe barrel and plunger rod;

FIG. 10 is a perspective view of an alternative embodiment of the backstop shown in FIG. 4;

FIG. 11 is a perspective view of an alternative embodiment of the plunger rod shown in FIG. 2; and

FIG. 12 is a perspective view of a further alternative embodiment of the plunger rod shown in FIG. 2.

### DETAILED DESCRIPTION OF THE PREFERRED EMBODIMENTS

Before describing the present disclosure in detail, it is to be understood that this disclosure is not limited to particularly exemplified apparatus, systems, methods, or process parameters that may, of course, vary. It is also to be understood that the terminology used herein is only for the purpose of describing particular embodiments of the present invention, and is not intended to limit the scope of the invention in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “port” includes one, two, or more ports.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the invention or claims.

Where possible, like numbering of elements have been used in various figures. Furthermore, multiple instances of an element and or sub-elements of a parent element may each include separate letters appended to the element number. For example two instances of a particular element “91” may be labeled as “91a” and “91b”. In that case, the element label may be used without an appended letter (e.g., “91”) to generally refer to instances of the element or any one of the elements. Element labels including an appended letter (e.g., “91a”) can be used to refer to a specific instance of the element or to distinguish or draw attention to multiple uses of the element. Furthermore, an element label with an appended letter can be used to designate an alternative design, structure, function, implementation, and/or embodiment of an element or feature without an appended letter. Likewise, an element label with an appended letter can be used to indicate a sub-element of a parent element. For instance, an element “12” can comprise sub-elements “12a” and “12b.”

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “connected,” and the like do not necessarily imply direct contact between the two or more elements.

Various aspects of the present devices, systems, and methods may be illustrated with reference to one or more examplary embodiments. As used herein, the term “examplary” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

In general, the present invention is directed to syringe assemblies that are designed to hold a dosage. The syringe assemblies are configured so that when they are subject to a change in pressure, such as when being transported by aircraft, the plunger rod is restrained from retracting out of the syringe barrel. However, the plunger rod can still be manually depressed when it is desired to dispense the dosage from the syringe barrel.

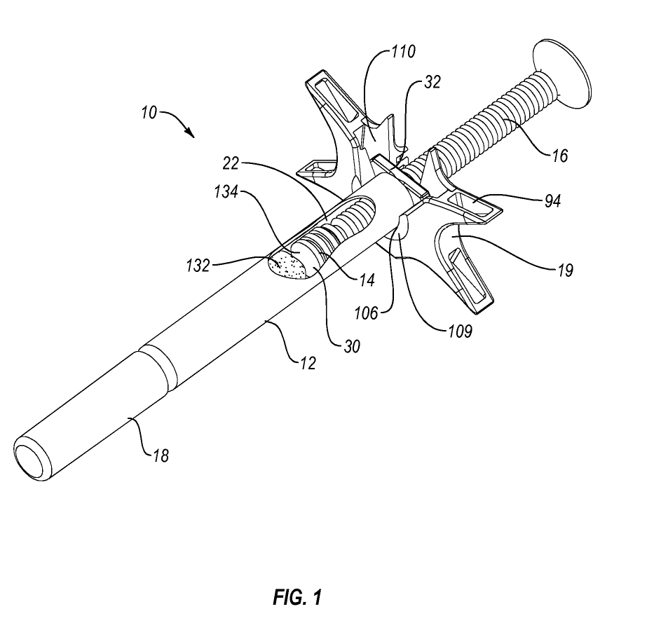

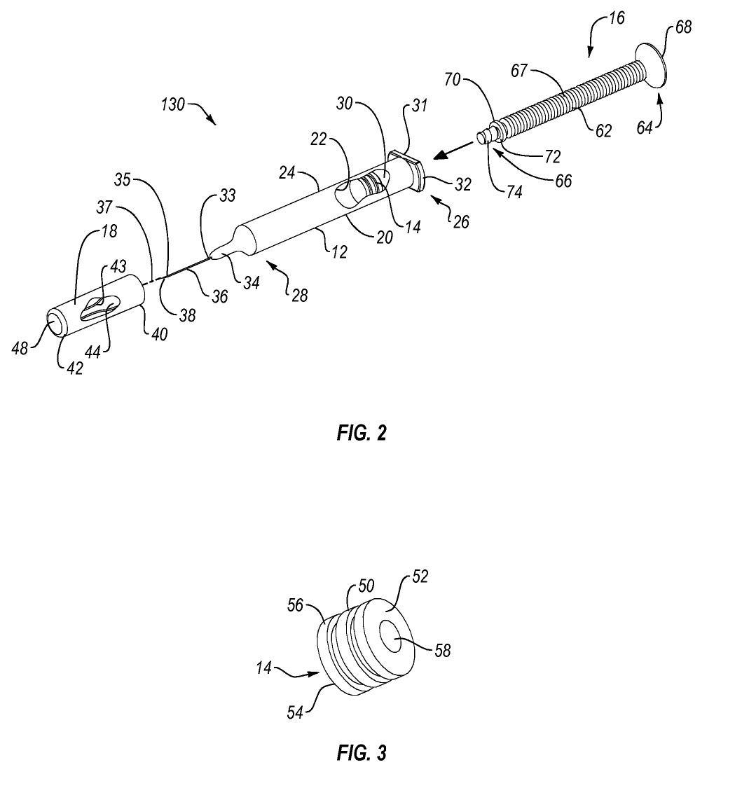

Depicted in FIG. 1 is one embodiment of a syringe assembly 10 incorporating features of the present invention. In general, syringe assembly 10 comprises a syringe barrel 12, a needle 36 (FIG. 2) extending from a distal end of syringe barrel 12, a needle shield 18 that covers needle 36, a stopper 14 that is received within syringe barrel 12, a plunger rod 16 that couples with stopper 14, and a backstop 19 that restrains movement of plunger rod 16 out of syringe barrel 12. The above elements will now be discussed in greater detail.

As depicted in FIG. 2, syringe barrel 12 comprises an elongated cylindrical body 20 having an interior surface 22 and an exterior surface 24 that longitudinally extend between a proximal end 26 and an opposing distal end 28. Interior surface 22 bounds a compartment 30 that extends between opposing ends 26 and 28. An opening 31 is formed at proximal end 26 and communicates with compartment 30. A flange 32 radially outwardly projects from proximal end 26. Flange 32 can encircle proximal end 26 or can project out from opposing sides of proximal end 26.

Projecting from body 20 at distal end 28 is an elongated stem 34. Stem 34 has a transverse cross section with a diameter that is smaller than the diameter of the transverse cross section of body 20. As discussed below, stem 34 is configured to receive and engage needle shield 18. Syringe barrel 12, or at least body 20, is typically made of a transparent or semi-transparent material such as clear plastic or glass. In other embodiments, however, body 20 can be opaque.

Projecting from stem 34 of syringe barrel 12 along a central longitudinal axis 37 of body 20 is tubular needle 36. Needle 36 has a proximal end 33 that communicates with compartment 30 of syringe barrel 12 and an opposing distal end 35 that terminates at a sharpened tip 38. Needle 36 is typically made of metal. In the embodiment depicted, needle 36 is permanently attached to syringe barrel 12 such as by being molded into stem 34 or by being attached by an adhesive. In alternative embodiments, needle 36 or the combination of needle 36 and stem 34 can be removably and/or mechanically attached to syringe barrel 12. For example, stem 34 and needle 36 can be formed with complementary halves of a luer-lock or snap-fit connection that permit fluid coupling therebetween. Other types of fluid connections can also be used.

As also depicted in FIG. 2, needle shield 18 has a first end 40 designed for receiving needle 36 and stem 34 of syringe barrel 12 and an opposing second end 42 that terminates at a terminal end face 48. Needle shield 18 has an interior surface 43 that bounds a cavity 44 that is open at first end 40. Cavity 44 is configured to receive needle 36 and stem 34 so that a friction tight engagement is formed between interior surface 43 and stem 34. When needle shield 18 is attached to syringe barrel 12 by the friction tight engagement, needle 36 is safely enclosed within cavity 44. Needle shield 18 typically has a portion comprised of a rigid plastic that prevents needle 36 from puncturing therethrough and a more flexible or elastomeric portion that engages with stem 34. In alternative embodiments, a mechanical connection can be used to secure needle shield 18 to syringe barrel 12. For example, needle shield 18 and stem 34 can be formed with complementary halves of a luer-lock connection that permit coupling therebetween. Other types of connection can also be used.

Slidably disposed within compartment 30 of syringe barrel 12 is stopper 14. As depicted in FIG. 3, stopper 14 has an annular side face 50 that extends between a proximal end face 52 and an opposing distal end face 54. Encircling and radially outwardly projecting from side face 50 are a plurality of lip seals 56. Lip seals 56 maintain a liquid tight seal against interior surface 22 (FIG. 2) of syringe barrel 12 as stopper 14 longitudinally moves within compartment 30. Accordingly, as stopper 14 advances towards distal end 28 of syringe barrel 12, any liquid within compartment 30 is dispensed out through needle 36. Centrally formed on proximal end face 52 is a threaded socket 58. Stopper 14 is typically comprised of an elastomeric material that enables a slidable, sealed engagement between stopper 14 and syringe barrel 12. Other materials that will achieve the desired functional operation can also be used.

Returning to FIG. 2, plunger rod 16 comprises an elongated shaft 62 that extends between a proximal end 64 and an opposing distal end 66. As better depicted in FIG. 9, shaft 62 typically has a circular transverse cross section. Encircling and radially outwardly projecting from shaft 62 along the length of shaft 62 are a plurality of teeth 67. Each tooth 67 has an asymmetrical transverse cross section that includes an annular proximal face 80 and an annular distal face 82 that intersect at an outside corner 84. Proximal face 80 is typically flat forming an inside angle θ₁ relative to a central longitudinal axis 78 of shaft 62 that is about 90°. In other embodiments, however, proximal face 80 could inwardly taper so that angle θ₁ is less than 90°, such as in a range between 40° and 90°. In still other embodiments, proximal face 80 could outwardly taper slightly such as in range between 90° and 110°. Other angles can also be used.

Distal face 82 has a frustoconical configuration forming an inside angle θ₂ relative to axis 78 that is typically in a range between about 20° and 80° with between 40° and 70° being more common. Again, other angles can be used. Typically there are more than 20 longitudinally spaced teeth 67 and more common more than 30, 40, or 60 teeth 67. In other embodiments, less than 20 teeth 67 can be used. Teeth 67 can butt directly against each other, e.g., distal face 82 of one tooth 67 can intersect with proximal face 80 of the adjacent tooth 67. In other embodiments, a gap 86 can be formed between adjacent teeth. Gap 86 is the longitudinal distance between where distal face 82 ends and the adjacent proximal face 80 starts. Any gap 86, however, is typically small, such as less than 2 mm and more commonly less than 1 mm or less than 0.5 mm. As a result of the tapering of distal face 82 in combination with any gap 86, an annular notch 87 is formed between each pair of adjacent teeth 67.

Returning to FIG. 2, disposed at proximal end 64 of shaft 62 is a thumb rest 68. Formed at distal end 66 of shaft 62 is an end plate 70. End plate 70 has a distal end face 72 from which a threaded stem 74 centrally projects. Threaded stem 74 is configured to thread into socket 58 of stopper 14 (FIG. 3) so that stopper 14 is securely connected to plunger rod 16 and so that distal end face 72 of plunger rod 16 can sit flush against proximal end face 52 of stopper 14. Plunger rod 16 is typically molded from a polymeric material, but other materials can also be used.

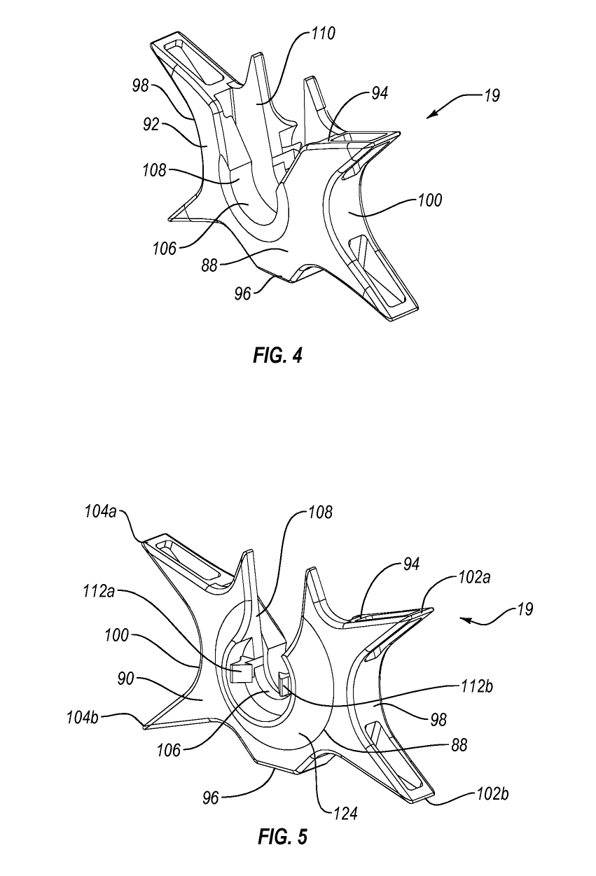

In one embodiment of the present invention, means are provided for coupling plunger rod 16 to syringe barrel 12 so that plunger rod 16 can be manually advanced into compartment 30 of syringe barrel 12 but is restrained from being manually retracted out of compartment 30 of syringe barrel 12. By way of example and not by limitation, depicted in FIGS. 4 and 5 is one embodiment of backstop 19. Backstop 19 comprises a base 88 having a proximal face 90 and an opposing distal face 92. Each of faces 90 and 92 extend between a top face 94 and an opposing bottom face 96 and between opposing side faces 98 and 100. A pair of spaced apart tapered fingers 102A and B radially outwardly project from side face 98 while a pair of spaced apart tapered finger 104A and B radially outwardly project from side face 100. A channel 106 having a substantially U-shaped configuration is recessed on top face 94 and longitudinally extends between proximal face 90 and distal face 92 of base 88. Channel 106 is bounded by an interior surface 108 of base 88. A slot 110 is recessed on interior surface 108 so as to be bounded between opposing faces 90 and 92 of base 88. As discussed below, slot 110 is configured to receive flange 32 of syringe barrel 12 (FIG. 2).

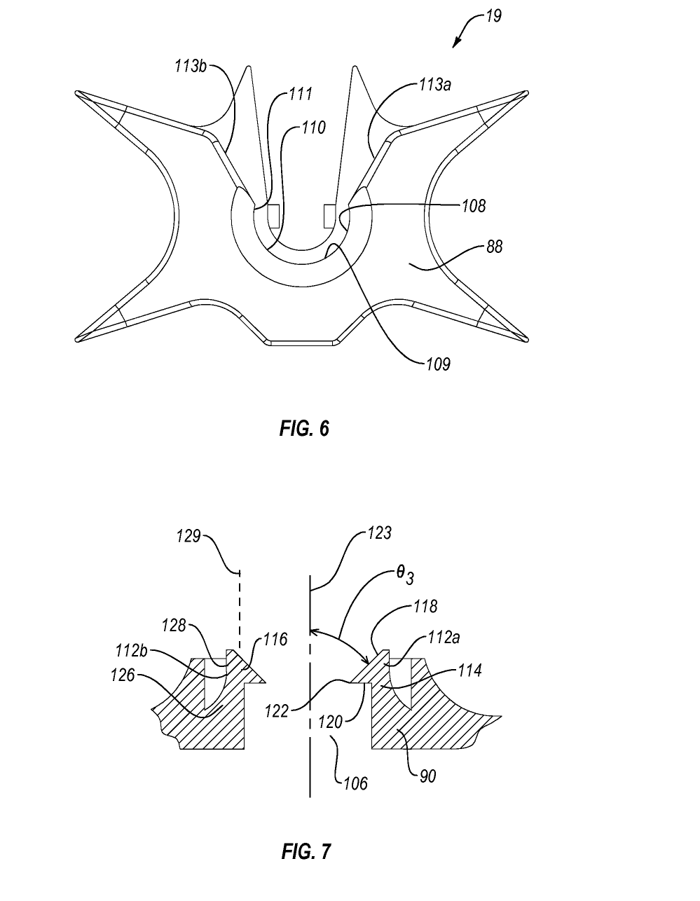

As depicted in FIG. 6, interior surface 108 of base 88 distal of slot 110 comprises a seat 109 having a having a C-shaped configuration with a slightly constricted mouth 111. Interior surface 108 also includes guides 113A and B that outwardly flare on opposing sides of mouth 111. Seat 109 is configured to receive syringe barrel 12 (FIG. 2). Mouth 111 is slightly smaller than the diameter of syringe barrel 12 so that when syringe barrel 12 is pushed into seat 109, seat 109 resiliently expands to allow syringe barrel 12 to pass through mouth 111 and then resiliently closes around syringe barrel 12 to snugly secure syringe barrel 12 to base 88. In alternative embodiments, seat 109 need not have a C-shaped configuration but could have a U-shaped configuration or rounded V-shaped configuration that does not expand when syringe barrel 12 is received therein.

As depicted in FIG. 5, outwardly projecting proximally from proximal face 90 of base 88 on opposing sides of channel 106 are a pair of pawls 112A and 112B. As better depicted in FIG. 7, each pawl 112 comprises a leg 114 having a first end 126 that connects to proximal face 90 and an opposing second end 128. A longitudinal axis 129 extends along the length of leg 114. Each pawl 112 also includes an asymmetrically tapered barb 116 that projects from second end 128 of leg 114 into channel 106. Each barb 116 has a proximal face 118 and an opposing distal face 120 that intersect at an outside corner 122. Relative to a central longitudinal axis 123 of channel 106, proximal face 118 is typically sloped at an angle θ₃ that is substantially the same as angle θ₂ of distal face 82 of teeth 67 (discussed above) or is within a range of +/−5° or 10° relative thereto. Accordingly, angle θ₃ is typically in a range between about 20° to about 80° with about 40° to about 70° being more common. Distal face 120 is typically disposed orthogonal to axis 123 or axis 129 of leg 114 but could be disposed in the same range of angles relative to axis 123 as angle θ₁ previously discussed above. Other angles can also be used.

Returning to FIG. 5, a guard 124 having a substantially C-shaped configuration outwardly projects from proximal face 90 of base 88 so as to partially encircle pawls 112. Guard 124 helps to prevent any outside item from striking pawls 112 which could break, damage or obstruct them. Guard 124 is depicted as tapering so as to have a frustoconical configuration but other designs can also be used.

During assembly, a pre-loaded syringe barrel 130 is provided, as depicted in FIG. 2. The pre-loaded syringe barrel 130 comprises syringe barrel 12, needle shield 18 secured to distal end 28, stopper 14 disposed within compartment 30 of syringe barrel 12, and a liquid dosage 132 (FIG. 1) disposed within compartment 30 between stopper 14 and needle shield 18. Dosage 132 can comprise a drug, a drug comparative, or a placebo, such as a saline solution. Often, although not always, an air bubble 134 (FIG. 1) can also be located with compartment 30 between stopper 14 and needle shield 18 or liquid dosage 132.

Once pre-loaded syringe barrel 130 is provided, plunger rod 16 can be advanced into compartment 30. Stem 74 of plunger rod 16 is then threaded into socket 58 (FIG. 3) of stopper 14 so as to secure stopper 14 to plunger rod 16. Care is taken to not substantially move stopper 14 within compartment 30 while stopper 14 is being secured to plunger rod 16 so as to avoid contamination of dosage 132. One example of how to attach plunger rod 16 to stopper 14 is disclosed in U.S. Provisional patent application Ser. No. 14/454,525, file Aug. 7, 2014 which is incorporated herein by specific reference. Other methods can also be used.

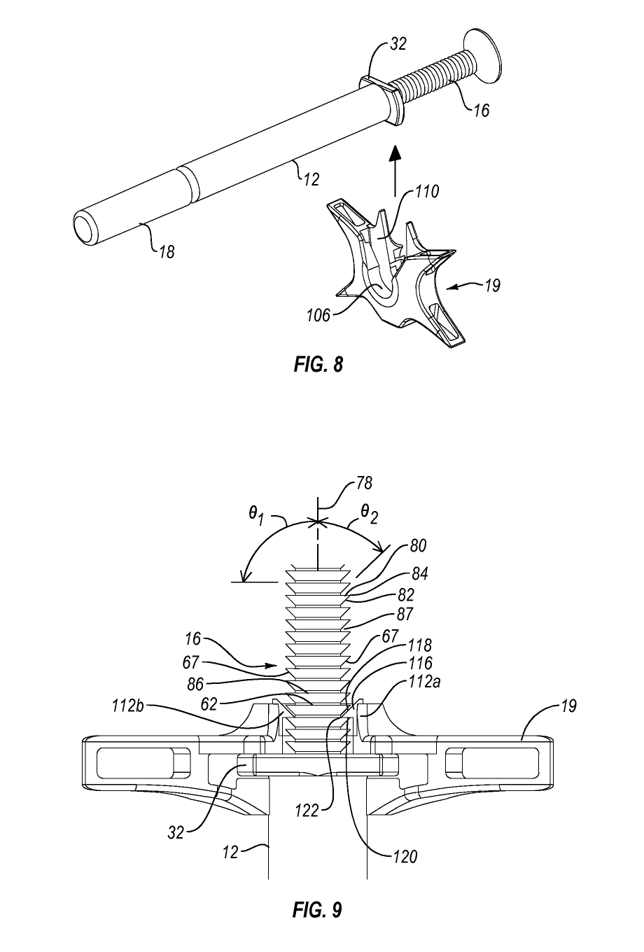

Once plunger rod 16 is secured to stopper 14, as depicted in FIG. 8, backstop 19 is secured to both syringe barrel 12 and plunger rod 16. This is accomplished by flange 32 of syringe barrel 12 being slid into slot 110 of backstop 19 so that syringe barrel 12 is received within channel 106, as depicted in FIG. 1. A variety of different mechanisms can be used for securing together syringe barrel 12 and backstop 19. For example, the connection can be a result of frictional engagement between backstop 19 and flange 32 and/or syringe barrel 12. As previously discussed, syringe barrel 12 can also snap-fit into seat 109 (FIG. 6) of backstop 19. In another embodiment, a retainer can be couple to top surface 94 of backstop 19, such as by a snap-fit connection, so that the retainer extends over syringe barrel 12 and thus holds backstop 19 to syringe barrel 12. For example, the combined backstop 19 and retainer could completely encircle or substantially encircle syringe barrel 12. In a like embodiment, backstop 19 can be comprised of two halves that couple together around syringe barrel 12 and/or flange 32 so as to completely or substantially encircle syringe barrel 12. In other embodiments, backstop 19 can be attached to syringe barrel 12 and/or flange 32 by welding, adhesive, fastener, clamp or the like.

In addition to backstop 19 coupling with syringe barrel 12 and/or flange 32, backstop 19 also engages with plunger rod 16. Specifically, as depicted in FIG. 9, as backstop 19 is being slid onto syringe barrel 12 and flange 32, barbs 116 of pawls 112 are slid into a notch 87 between adjacent teeth 67. In this configuration, pawls 112 and teeth 67 combined to form a ratchet that only permits plunger rod 16 to substantially move in one direction relative to backstop 19. Specifically, plunger rod 16 can be advanced into compartment 30 (FIG. 1) of syringe barrel 12 for use in dispensing dosage 132 but is restrained by backstop 19 from being pulled or pushed out of compartment 30. More specifically, distal face 82 of teeth 67 and proximal face 118 of pawls 112 are tapered, such as in the angles discussed above, so that when plunger rod 16 is pressed into compartment 30, distal face 82 of a tooth 67 rides against and radially outwardly pushes barbs 116 of pawls 112 so as to cause legs 114 (FIG. 7) to resiliently flex away from plunger rod 16.

Distal face 82 of tooth 67 continues to ride against proximal face 118 of barbs 116 until outside corner 122 of barb 116 passes over outside corner 84 of the tooth 67. Flexed legs 114 then resiliently force barbs 116 back into the next notch 87. The process is then repeated for the next tooth 67 until plunger rod 16 is advanced to the desired depth into compartment 30 (FIG. 1). Those skilled in the art will appreciate that proximal face 118 and distal face 82 can be formed in a variety of different angles and that the angles can be complementary or different from each other. Faces 118 and 82 simply need to be sloped so that teeth 67 radially outwardly press pawls 112 causing barbs 112 pass over teeth 67 as plunger rod 16 is advanced into compartment 30.

In contrast to proximal face 118, distal face 120 of barbs 116 are configured to engage proximal face 80 of teeth 67 to restrain plunger rod 16 from being pulled or pushed out of compartment 30. This is because faces 120 and 80 are orientated so that when face 80 of teeth 67 pushes against face 120 of barbs 116, there is no lateral force or insufficient lateral force to push pawls 112 laterally so that teeth 116 can pass around barbs 116. In the embodiment depicted, this is accomplished by both faces 120 and 80 being disposed in planes that are substantially parallel to each other and perpendicular to the longitudinal axis 129 of legs 114 (FIG. 7). This orientation also typically happens to be perpendicular to central longitudinal axis 78 of plunger rod 16. As discussed above, there are other orientation that faces 120 and 80 can be disposed that will still cause pawls 112 to engage teeth 67 and prevent the withdrawal of plunger rod 16.

In view of the foregoing, backstop 19 interacts with syringe barrel 12 and plunger rod 16 to permit plunger rod 16 to advance into syringe barrel 12 but restrains plunger rod from being pulled or pushed out of syringe barrel 12. Restraining the movement of plunger rod 16 out of syringe barrel 12 is helpful to prevent contamination of dosage 132 within compartment 30. For example, as depicted in FIG. 1, interior surface 22 of syringe barrel 12 proximal of stopper 14 is not sterile. Thus, if stopper 14 moves proximally within syringe barrel 12 a sufficient distance, dosage 132 within syringe barrel 12 can contact the non-sterile surface and thus become contaminated and unfit for use. This movement of stopper 14 can result from plunger rod 16 being manually pulled proximally when attached to stopper 14. Alternatively, because compartment 30 often includes gas bubble 134, as discussed above, when syringe assembly 10 is subject to a reduced atmospheric pressure, such as when syringe assemblies 10 are being transported by aircraft or even when potentially transported by land but at high elevations, gas bubble 134 can expand in the reduced atmospheric pressure. As gas bubble 134 expands, it applies a force to stopper 14 trying to push stopper 14 and plunger rod 16 proximally out of syringe barrel 12. Such movement could result in dosage 132 contacting the non-sterile surface of syringe barrel 12.

Based on the above, one of the benefits of syringe assembly 10 is that backstop 19 restrains proximal movement of stopper 14 either as a result of an unwanted or unintentional exterior force being applied to plunger rod 16 or as a result of a pressure differential between compartment 30 and the surrounding environment. As a result, the probability of dosage 132 becoming contaminated is minimized.

It is appreciated that backstop 19 may not preclude all proximal movement of plunger rod 16 and stopper 14 relative to syringe barrel 12. For example, if notches 87 between adjacent teeth 67 are larger than barbs 116 that are received therein, plunger rod 16/stopper 14 may be free to slide proximally and distally along the open space within the specific notch 87. In this embodiment, the permitted movement should be less than the distance that stopper 14 must move before dosage 132 can reach the non-sterile surface of barrel 12. Typically, any such free movement is less than 2 mm and more commonly less than 1 mm or less than 0.5 mm.

In the embodiment depicted, backstop 19 is configured to preclude any further proximal movement of plunger rod 16 once barbs 116 contact proximal face 80 of a tooth 67. In other embodiments, however, barbs 116 and teeth 67 could be configured so that when a sufficient manual force is applied to plunger rod 16, plunger rod 16 can be intentionally moved proximally as a result of lateral movement of barbs 16 around teeth 67. This could occur as a result of providing a slight slope to distal face 120 of barbs 116 and/or proximal face 80 of teeth 67, as discussed above. In this embodiment, however, the required force to move plunger rod 16 proximally needs to be greater than forces that are typically applied to plunger rod 16 that can produce unwanted proximal movement of plunger rod 16. For example, the force would need to be greater than the maximum force that stopper 14 could be subject to as a result of a change in atmospheric pressure that syringe assembly 10 is exposed to during normal transportation. Thus, in this embodiment, plunger rod 16 should only be able to move proximally when a manual pulling force greater than what could be applied to plunger rod 16 by environmental conditions is applied to plunger rod 16. Backstop 19 also achieves other unique benefits. For example, because backstop 19 still permits plunger rod 16 to move distally for the dispensing of dosage 132, it is not necessary to remove backstop 19 after syringe assembly 10 has been transported to its final destination and it is desired to dispense dosage 132. Rather, backstop 19 functions as an enlarged finger flange for use in gripping syringe barrel 12 during dispensing of dosage 132. That is, during use an operator's middle and index fingers rest against backstop 19 while plunger rod 16 is advanced under a force applied by a thumb of the operator. Because backstop 19 functions as a finger flange, flange 32 can be formed smaller than normal during the original manufacture of syringe barrel 12, thereby decreasing the cost of syringe barrel 12. Furthermore, because some addition force is required move plunger rod 16 distally during dispensing of dosage 132, backstop 19 also functions to help prevent unwanted dispensing of dosage 132 from syringe barrel 12 and enables greater accuracy in potentially dispensing select quantities of dosage 132 from syringe barrel 12. In addition, as a result of the spaced, outwardly projecting legs 102 and 104 (FIG. 5) of backstop 19, syringe assemblies 10 can rest in an inclined orientation on a table top or other surface for easy grasping or other manipulation. In other embodiments, backstop 19 could be removed from syringe barrel 12 prior to dispensing dosage 132. In this embodiment, flange 32 would function as a finger flange for holding syringe barrel 12 during dispensing.

Backstop 19 also has the unique benefit in that it can be used on syringe barrel 12 for any desired size of dosage 132 within syringe barrel 12. That is, independent of how far in or out plunger rod 16 is disposed within syringe barrel 12, backstop 19 can still be attached to restrain movement of plunger rod 16. Depending on the configuration, a single sized backstop 19 can also be used with a variety of syringe barrels having different configurations. Thus, a single sized backstop 19 has greater universal use than a conventional packaging case that is used to prevent movement of a plunger rod relative to a syringe barrel. Likewise, backstop 19 eliminates the need to use conventional enlarged, reinforced packaging cases.

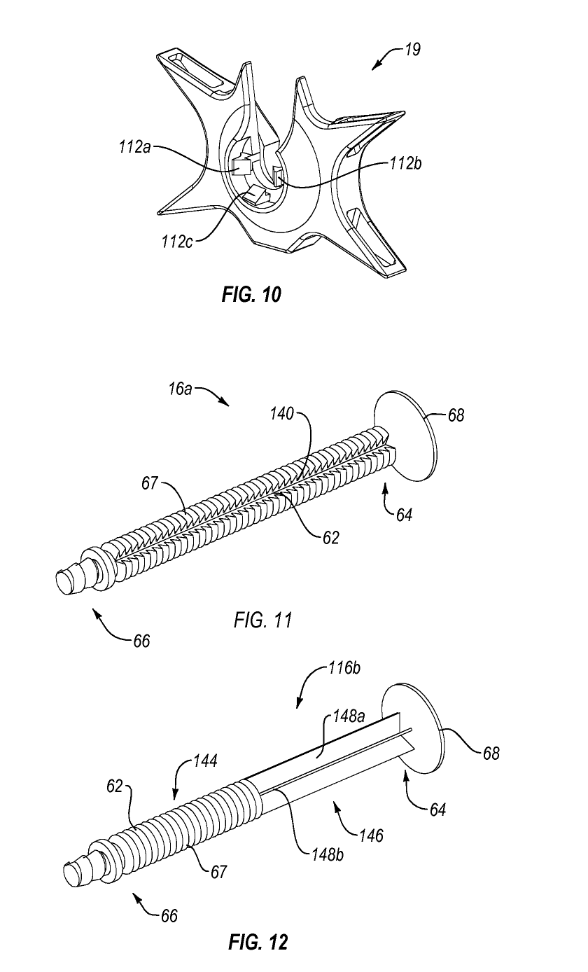

It is also appreciated that backstop 19 can have a variety of different configurations. For example, legs 102 and 104, as depicted in FIG. 5, can be eliminated. In this embodiment, base 88 of backstop 19 could have a substantially circular, square, rectangular, polygonal, or other configuration. Furthermore, in contrast to having both barbs 116 of pawls 112 being concurrently received within the same notch 87 as depicted in FIG. 9, it is appreciated that pawls 112a and b could be of different configurations so that they are concurrently received within different notches 87 or at different locations along notches 87. Furthermore, in the embodiment depicted, backstop 19 is shown as having two pawls 112a and b that engage with teeth 67. In other embodiments, backstop 19 may have a single pawl 112, three pawls 112a, 112b, and 112c (as depicted in FIG. 10), or could have four or more separate pawls 112 for engaging with teeth 67.

It is likewise appreciated that plunger rod 16 can also have a variety of different configurations. By way of example and not by limitation, depicted in FIG. 11 is a plunger rod 16a wherein like elements between plunger rod 16a and 16 are identified by like reference characters. Plunger rod 16 and 16a are substantially identical except that plunger rod 16a has elongated slots 140 that extend along the length of shaft 62 so as to pass through teeth 67. Slots 140 form a surface which better enables mechanically grasping plunger rod 16a. This mechanical grasping of plunger rod 16a is used during the attachment of plunger rod 16a to stopper 14 as described in U.S. Provisional patent application Ser. No. 14/454,525, which was previously incorporated by reference. In different embodiments, there can be two, three, four, or more radially spaced apart slots 140 extending along the length of shaft 62. Slots 140 can divide teeth 67 into a first plurality of teeth, a second plurality of teeth, a third plurality of teeth and so on depending upon how many separating slots 140 are formed. Each of the plurality of teeth 67 or the like disclosed herein can also be referred to as a rack of teeth. Slots 140 typically extend linearly along the length of shaft 62 and can form an inside right angle or other inside angles. In other embodiments, it is not necessary that slots 140 extend the full length of shaft 62. For example, slots may be disposed at proximal end 64 and extend less than ¾, ½, or ⅓ of the length of shaft 62.

Depicted in FIG. 12, is an alternative embodiment of a plunger rod 16b. Again like elements between plunger rod 16 and 16b are identified by like reference characters. Plunger rod 16b comprises a lower shaft portion 144 disposed at distal end 66 and an upper shaft portion 146 disposed at proximal end 64. Lower shaft portion 144 comprises shaft 62 having encircling teeth 67 as previously discussed herein. Upper shaft portion 146 has a substantially X-shaped transverse cross section that is comprised of two elongated rails 148a and b that bisect each other at right angles along their length. Again, upper shaft portion 146 is configured to facilitate mechanical engagement with plunger rod 16B. In one embodiment, upper shaft portion 146 is at least 60% and more commonly at least 80% or 100% of the length of lower shaft portion 144.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A syringe assembly comprising:
a syringe barrel comprising a tubular body that bounds a compartment extending between a proximal end and an opposing distal end and a flange that radially outwardly projects from the proximal end of the body;
a stopper movably disposed with the compartment of the syringe barrel;
a plunger rod comprising a shaft having a proximal end and an opposing distal end and a plurality of teeth disposed on the shaft, the distal end of the shaft being secured to the stopper; and
means for coupling the plunger rod to the syringe barrel so that the plunger rod can be manually advanced into the compartment of the syringe barrel but is restrained from being manually retracted out of the compartment of the syringe barrel.

2. The syringe assembly as recited in claim 1, wherein the means for coupling comprises a backstop that engages the flange of the syringe barrel and at least a portion of the plurality of teeth of the plunger rod.

3. The syringe assembly as recited in claim 1, wherein the means for coupling comprises a backstop, the backstop comprising:
a base having a proximal face and an opposing distal face with a channel extending between the opposing faces, a slot being recessed on an interior surface bounding at least a portion of the channel, a portion of the tubular body of the syringe barrel being received within the channel so that at least a portion of the flange is captured within the slot; and
a resiliently flexible first pawl disposed on the base and engaging at least one of the plurality of teeth of the plunger rod, the first pawl and the plurality of teeth forming a ratchet.

4. The syringe assembly as recited in claim 3, further comprising:
the flexible first pawl comprising a leg that projects from the base and an asymmetrical tapered barb mounted on the end thereof; and
each of the plurality of teeth of the plunger rod having an asymmetrical transverse cross section.

5. The syringe assembly as recited in claim 3, wherein the backstop further comprises a resiliently flexible second pawl disposed on the base and engaging at least one of the plurality of teeth of the plunger rod, the second pawl and the plurality of teeth forming a ratchet.

6. The syringe assembly as recited in claim 1, wherein each of the plurality of teeth of the plunger rod radially encircle the shaft.

7. The syringe assembly as recited in claim 1, wherein the plurality of teeth of the plunger rod comprise:
a first plurality of teeth disposed longitudinally along the length of the shaft;
a second plurality of teeth disposed longitudinally along the length of the shaft and radially spaced apart from the first plurality of teeth so that an elongated first slot is formed therebetween; and
a third plurality of teeth disposed longitudinally along the length of the shaft and radially spaced apart from the second plurality of teeth so that an elongated second slot is formed therebetween.

8. A syringe assembly comprising:
a syringe barrel comprising a tubular body that bounds a compartment extending between a proximal end and an opposing distal end and a flange that radially outwardly projects from the proximal end of the body;
a stopper movably disposed with the compartment of the syringe barrel;
a plunger rod comprising a shaft having a proximal end and an opposing distal end and a plurality of teeth disposed on the shaft, the distal end of the shaft being secured to the stopper; and
a backstop that engages the flange of the syringe barrel and at least a portion of the plurality of teeth of the plunger rod.

9. The syringe assembly as recited in claim 8, wherein the backstop and the plurality of teeth combine to form a ratchet that enables the plunger rod to be manually advanced into the compartment of the syringe barrel but restrains the plunger rod from being manually retracted out of the compartment of the syringe barrel.

10. The syringe assembly as recited in claim 9, wherein the backstop is manually removable from the syringe barrel.

11. The syringe assembly as recited in claim 8, wherein the backstop comprises:
a base having a proximal face and an opposing distal face with a channel extending between the opposing faces, a slot being recessed on an interior surface bounding at least a portion of the channel, a portion of the tubular body of the syringe barrel being received within the channel so that at least a portion of the flange is captured within the slot; and
a resiliently flexible first pawl being disposed on the base and engaging at least one of the plurality of teeth of the plunger rod, the first pawl and the plurality of teeth forming a ratchet.

12. The syringe assembly as recited in claim 11, wherein the channel has a substantially U-shaped, V-shaped or C-shaped configuration.

13. The syringe assembly as recited in claim 11, further comprising a resiliently flexible second pawl being disposed on the base and engaging at least one of the plurality of teeth of the plunger rod.

14. The syringe assembly as recited in claim 8, wherein the plurality of teeth of the plunger rod comprise:
a first plurality of teeth disposed longitudinally along the length of the shaft;
a second plurality of teeth disposed longitudinally along the length of the shaft and radially spaced apart from the first plurality of teeth so that an elongated first slot is formed therebetween; and
a third plurality of teeth disposed longitudinally along the length of the shaft and radially spaced apart from the second plurality of teeth so that an elongated second slot is formed therebetween.

15. The syringe assembly as recited in claim 8, wherein the shaft of the plunger rod comprises:
a lower portion having the plurality of teeth formed thereon; and
am upper portion having a substantially X-shaped shaped transverse cross section.

16. A method for assembling a syringe, the method comprising:
threading a distal end of a plunger rod into a stopper located within a compartment of a syringe barrel, a liquid dosage being disposed within a portion of the compartment extending between the stopper and a distal end of the syringe barrel; and
securing a backstop to a flange of the syringe barrel so that a pawl of the backstop engages a rack of teeth of the plunger rod, the pawl and the rack of teeth forming a ratchet that permits the plunger rod to be manually advanced into the compartment of the syringe barrel but restrains the plunger rod from being manually retracted out of the compartment of the syringe barrel.

17. The method as recited in claim 16, further comprising dispensing the liquid dosage from the syringe barrel while the backstop remains secured to the syringe barrel by advancing the plunger rod within the compartment of the syringe barrel.

18. The method as recited in claim 17, wherein the step of dispensing comprises using the backstop as a finger flange against which an operator's fingers rest while the plunger rod is advanced under a force applied by a thumb of the operator.

19. The method as recited in claim 17, wherein the step of dispensing comprises advancing the plunger rod into the compartment of the syringe barrel so that the pawl flexes outwardly and then resiliently rebounds as the pawl passes over a tooth of the rack of teeth.

20. The method as recited in claim 16, wherein the step of securing comprises advancing a portion of the syringe barrel into a channel of the backstop so that the flange of the syringe barrel in received within a slot recessed on the backstop.
